SEQUENCE LISTING (4 sequences)

SEQ ID NO: 1 - protein, 269 aa, Thermomyces lanuginosus
EVSQDLFNQFNLFAQYSAAAYCGKNNDALAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVLVTRNDIVKIEGIDATGGNNQPNIPDIPAHLWYFGLIGTCL

SEQ ID NO: 2 - protein, 269 aa, synthetic construct
DVSADLLNQFKLFAQYSAAAYCGRNNDAPAGTNITCSANACPLVEAADATFLYSFENSGVGDVTGFLAVDNTNKLIVLSFRGSRSIENWITNLNFDLVDINDICSGCRGHDGFTSSWRSVADTLRQAVDDAVAAHPDYKVVVTGHSLGGALATLAGADLRNNGYDVDLYTYGAPRVGNRAFAEFITVQTGGTLYRVTHTNDVVPRLPPRQFGYSHPSPEYWIKSGTLVPVRRRDIVKIEGIDATGGNNQNNTPDIPAHLWYFGLIGTCI

SEQ ID NO: 3 - protein, 462 aa, Moesziomyces antarcticus
MRVSLRSITSLLAAATAAVLAAPAAETLDRRAALPNPYDDPFYTTPSNIGTFAKGQVIQSRKVPTDIGNANNAASFQLQYRTTNTQNEAVADVATVWIPAKPASPPKIFSYQVYEDATALDCAPSYSYLTGLDQPNKVTAVLDTPIIIGWALQQGYYVVSSDHEGFKAAFIAGYEEGMAILDGIRALKNYQNLPSDSKVALEGYSGGAHATVWATSLAESYAPELNIVGASHGGTPVSAKDTFTFLNGGPFAGFALAGVSGLSLAHPDMESFIEARLNAKGQRTLKQIRGRGFCLPQVVLTYPFLNVFSLVNDTNLLNEAPIASILKQETVVQAEASYTVSVPKFPRFIWHAIPDEIVPYQPAATYVKEQCAKGANINFSPYPIAEHLTAEIFGLVPSLWFIKQAFDGTTPKVICGTPIPAIAGITTPSADQVLGSDLANQLRSLDGKQSAFGKPFGPITPP

SEQ ID NO: 4 - protein, 342 aa, Moesziomyces antarcticus
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

(19) United States
(12) Patent Application Publication
(10) Pub. No.: US 2025/0257375 A1
(43) Pub. Date: Aug. 14, 2025

(54) PRODUCTION OF FATTY ACID ALKYL ESTERS

(71) Applicant: Novozymes A/S, Bagsvaerd (DK)

(72) Inventors: Rasmus Boeg HANSEN, Frederiksberg (DK); Per Munk Nielsen, Hilleroed (DK)

(73) Assignee: Novozymes A/S, Bagsvaerd (DK)

(21) Appl. No.: 18/857,436

(22) PCT Filed: Apr. 27, 2023
(86) PCT No.: PCT/EP2023/061073

§ 371 Date: Oct. 16, 2024

(30) Foreign Application Priority Data
Apr. 29, 2022 (EP) 22170819.1

(51) Int. Cl. C12P 7/649; C12N 9/20

(52) U.S. Cl. CPC C12P 7/649; C12N 9/20; C12Y 301/01003

(57) ABSTRACT

The present invention provides a process for producing fatty acid alkyl esters. The process comprises providing a substrate comprising triglycerides, diglycerides, monoglycerides, free fatty acids, or any combination thereof, and reacting the substrate with an enzyme composition comprising an sn-1,3 position lipase and an sn-2 position lipase to produce fatty acid alkyl esters.

### REFERENCE TO A SEQUENCE LISTING

This application contains a Sequence Listing in computer readable form, which is incorporated herein by reference.

### FIELD OF THE INVENTION

The present invention relates to a process for producing fatty acid alkyl esters from substrate using an sn-1,3 position lipase and an sn-2 position lipase combination which favours the conversion of free fatty acids and/or triglyceride to fatty acid alkyl esters.

### BACKGROUND ART

Biodiesel, generally classified as mono-alkyl esters of fats and oils, has become more attractive recently because of its environmental benefits. Although biodiesel is at present successfully produced chemically (using e.g., NaOH and/or sodium methoxide as catalyst), there are several associated problems to restrict its development, such as pre-processing of oil due to high contents of free fatty acids, removal of chemical catalyst from ester and glycerol phase and removal of inorganic salts during glycerol recovery.

The disadvantages caused by chemical catalysts are largely prevented by using lipolytic enzymes as the catalysts and in recent years interest has developed in the use of lipases with or without immobilization in transesterification for the production of biodiesel.

Fungal esterases may be used in the enzymatic production of esters, where they may replace catalysts like mineral acid (e.g., sulphuric acid, hydrogen chloride, and chlorosulfonic acid), amphoteric hydroxides of metals of groups I, II, III, and IV, and others. The use of enzymes for ester synthesis has been described in the prior art, in particular enzymes classified in EC 3.1.1 Carboxylic ester hydrolases according to Enzyme Nomenclature (Recommendations of the Nomenclature Committee of the International Union of Biochemistry and Molecular Biology, 1992 or later).

WO 88/02775 discloses lipases A and B from Candida antarctica. It states that C. antarctica lipase B (CALB) is more effective for ester synthesis.

Cutinases are lipolytic enzymes capable of hydrolyzing the substrate cutin. Cutinases are known from various fungi (P. E. Kolattukudy in “Lipases”, Ed. B. Borgström and H. L. Brockman, Elsevier 1984, 471-504). The amino acid sequence of a cutinase from Humicola insolens has been published (U.S. Pat. No. 5,827,719).

It is well known that excessive short-chain alcohols such as methanol might inactivate lipase seriously. However, at least three molar equivalents of methanol based on triglycerides are required for the complete conversion of the oil to its corresponding methyl ester. Du et al. (Biotechnol. Appl. Biochem. 2003, 38:103-106) studied the effect of molar ratio of oil/methanol comparatively during non-continuous batch and continuous batch operation.

To avoid inactivation of the lipases the methanol concentration has been kept low by step-wise addition of methanol throughout the reaction (Shimada et al. J Mol. Catalysis Enzymatic, 2002, 17:133-142; Xu et al. 2004, Biocat. Biotransform. 22:45-48).

Boutur et al. (J. Biotechnol. 1995, 42:23-33) reported a lipase from Candida deformans which were able to catalyse both alcoholysis of triglyceride (TG) and esterification of free fatty acids (FFA), but not under the same reaction conditions. Under the conditions described by Boutur et al. only the esterification was catalysed.

In order to obtain a more economic production of fatty acid ethyl esters for biodiesel, there is a need for a faster conversion of fats and oils to their corresponding methyl esters and a higher yield in said conversion.

### SUMMARY OF THE INVENTION

The present invention relates to a process for producing fatty acid alkyl esters. The process comprises steps of: a) providing a substrate comprising primarily triglycerides, diglycerides, monoglycerides, free fatty acids, or any combination thereof, and b) reacting the substrate with an enzyme composition comprising an sn-1,3 position lipase and an sn-2 position lipase to produce fatty acid alkyl esters.

The present invention also relates to an enzyme composition for producing fatty acid alkyl esters comprising an sn-1,3 position lipase and an sn-2 position lipase.

The present invention uses of combination of un-specific lipase which also catalyze reactions at sn-2-position having a low specific activity. The un-specific lipase can surprising boot the reaction rate and conversion by substituting part of the fast sn-1,3 position lipase.

These and still other objectives and advantages of the present invention will be apparent from the description which follows. In the detailed description below, preferred embodiments of the invention will be described in reference to the accompanying drawings. These embodiments do not represent the full scope of the invention. Rather the invention may be employed in other embodiments. Reference should therefore be made to the claims herein for interpreting the breadth of the invention.

### Definitions

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a step” includes reference to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition or are included only in amounts which are small enough so as to have no deleterious effect on the composition.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value and can include a range of plus or minus two standard deviations around the stated value.

Likewise, reference to a gene or polypeptide that is “derived from” another gene or polypeptide X, includes the gene or polypeptide X.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 percent to about 20 percent should be interpreted to include not only the explicitly recited concentration limits of 1 percent to about 20 percent, but also to include individual concentrations such as 2 percent, 3 percent, 4 percent, and sub-ranges such as 5 percent to 15 percent, 10 percent to 20 percent, etc.

Lipid: The term “lipid” refers to phospholipids and their derivatives, triglycerides and derivatives, sterols, stanols, cholesterol, sphingolipids, ceramides, fatty acids, fatty alcohols, glycolipids, proteolipids, lipopolysaccharides, ether-lipids, polar and non-polar lipids and derivatives thereof.

Esterification: The term “esterification” as used herein, refers to a reaction for combining an organic acid such as a fatty acid with any alcohol or polyol such as a glycerol.

Hydrolysis: The term “hydrolysis” as used herein, refers to the reaction of water with an ester to produce an acid and an alcohol.

Alcoholysis: The term “alcoholysis” as used herein, refers to the reaction of an ester with a monohydric alcohol, such as ethanol, butanol, or polyhydric alcohol as glycerol, to produce an ester with a different alkyl group.

Acidolysis: The term “acidolysis” as used herein, refers to the reaction of an ester with an acid leading to the exchange of acyl groups.

Interesterification: The term “interesterification” as used herein, refers to the reaction of a first ester with a second ester leading to a mix up between the acyl and the alcohol moieties.

Transesterification: The term “transesterification” as used herein, refers to any of the following reactions: alcoholysis, acidolysis and interesterification.

Synthesis: The term “synthesis” or “synthesis of fatty acids” as used herein, refer to covalently binding a fatty acid at the sn-2 position of a glyceride, preferably by a one-step reaction selected from any one of the following reactions: esterification, interesterification, alcoholysis, acidolysis, transesterification.

The terms “alkyl” or “alkyl group” is to be construed according to its broadest meaning, to describe a univalent aliphatic compound comprising hydrocarbons.

The terms “glycerol derivatives” and “glycerides” are interchangeably used herein to describe esters, ethers and other derivatives of glycerol in which at least one of the hydrogens, of any of the hydroxyl group attached to the CI, C2 or C3 carbons, is substituted. Examples of glycerol derivatives are: tristearoylglycerol (or tri-Ostearoyl glycerol or glycerol tristearate, or glyceryl tristearate); I,3-benzylideneglycerol (or 1,3-0-benzylideneglycerol); and glycerol 2-phosphate (or 2-phosphoglycerol) among others. If the substitution is on a carbon atom, rather than on the oxygen of the hydroxyl group than the compound may be considered as a derivative of glycerol (e.g., 1,2,3-nonadecanetriol for C16H33CHOH—CHOH—CH2OH, which may be also considered as 1-C-hexadecyl glycerol). The term “glycerol” as used herein is intended to encompass glycerol derivatives.

Lipase: The terms “lipase”, “lipase enzyme”, “lipolytic enzyme”, “lipid esterase”, “lipolytic polypeptide”, and “lipolytic protein” refers to an enzyme in class EC3.1.1 as defined by Enzyme Nomenclature. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50). For purposes of the present invention lipase activity (i.e. the hydrolytic activity of the lipase) may be determined with a pNP assay using substrates with various chain length as described in the “Materials & Methods”-section.

Parent or parent lipase: The term “parent” or “parent lipase” means a lipase to which an alteration is made to produce the enzyme variants. The parent lipase may be a naturally occurring (wild-type) polypeptide but may also be a variant and/or fragment thereof. In preferred embodiments, the parent lipase may be the ones shown in SEQ ID NOs: 1, 2, 3 or 4.

Sequence identity: The relatedness between two amino acid sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Substrates: Suitable substrates for production of fatty acid alkyl esters in accordance with the present invention are a broad variety of vegetable oils and fats; rapeseed and soybean oils are most commonly used, though other crops such as mustard, sunflower, canola, coconut, hemp, palm oil and even algae show promise. The substrate can be of crude quality or further processed (refined, bleached and deodorized). Also, animal fats including tallow, lard, poultry, marine oil as well as waste vegetable and animal fats and oil, commonly known as yellow and brown grease can be used. The suitable fats and oils may be pure triglyceride or a mixture of triglyceride and free fatty acids, commonly seen in waste vegetable oil and animal fats. The substrate may also be obtained from vegetable oil deodorizer distillates. The type of fatty acids in the substrate comprises those naturally occurring as glycerides in vegetable and animal fats and oils. These include oleic acid, linoleic acid, linolenic acid, palmetic acid, steric acid, and lauric acid to name a few. Minor constituents in crude vegetable oils are typically phospholipids, free fatty acids and partial glycerides i.e., mono- and diglycerides. When used herein the phrase “fatty acid residues” refers to fatty acids, either free or esterified as in triglycerides, diglycerides, monoglycerides or fatty acid alkyl esters.

Biodiesel: Fatty acid alkyl esters (FAAE) of short-chain alcohols, such as fatty acid methyl esters (FAME) and fatty acid ethyl esters (FAEE) are also called biodiesel, because they are used as an additive to fossil diesel. Biodiesel constitutes an increasingly important additive or substitute for diesel fuels based on fossil oil because it is produced from renewable resources.

Alcohol: Alcohol used in the process of the invention is preferably a short-chain, branched or linear, alcohol having 1 to 5 carbon atoms (C₁, C₂, C₃, C₄, or C₅, “a lower alcohol”) and mixtures thereof. Preferred lower alcohols are methanol, ethanol, propanol or mixtures thereof. The alcohol content is preferably less than 4.0, 3.5, 3.0, 2.5, 2.0, 1.5 or 1.0 molar equivalents to the amount of fatty acids in the reaction mixture (free and glyceride bound fatty acids). The alcohol may be added stepwise (such as in 1, 2, 3, 4, 5, 6, 7, 8, 9, 10 or more steps) and/or continuously to the reaction mixture.

Fatty Acid Feedstock: The term “fatty acid feedstock” or “oils and/or fats” or “vegetable oil feedstock” is defined herein as a substrate comprising fatty acid derivatives. The substrate may comprise fatty acid alkyl esters, triglyceride, diglyceride, monoglyceride, free fatty acid or any combination thereof. Any oils and fats of vegetable or animal origin comprising fatty acids may be used as substrate for producing fatty acid alkyl esters in the process of the invention. Also, fatty acid feedstock consisting substantially of fatty acid alkyl esters is suitable as feedstock (biodiesel feedstock) for the present invention. Preferably, the free fatty acid content of the fatty acid feedstock is above 0.25%, above 0.30%, above 0.35%, above 0.50%, above 0.75%, above 1.0%, above 5.0%, above 10.0%, above 15.0%, above 20.0%, above 25.0%, above 30.0%, above 40%, or even above 50.0%.

The fatty acid feedstock may be oil selected from the group consisting of: microbial oil, algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, distillers' corn oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, or any combination thereof.

The fatty acid feedstock may be fat selected from the group consisting of animal fat, including tallow from pigs, beef and sheep, lard, chicken fat, fish oil, or any combination thereof.

The fatty acid feedstock may be crude, refined, bleached, deodorized, degummed, or any combination thereof.

Food quality oils and fats are expensive, and therefore, waste and by-products from their processing as well as non-food grade oils and fats have become increasingly attractive feedstock for producing fatty acid alkyl ester. Soap stock is the fraction of oil obtained in an oil refinery by treating the oil with a base to convert free fatty acids to soaps (e.g., sodium soaps). The soap stock usually contains a fraction of glycerides beside the soaps. Acid oil is the by-product from the oil refinery produced by acidification of soap stock to solubilize the soaps. It mainly contains free fatty acids (FFA) and acylglycerols. Distillates like Palm Fatty Acid Distillate (PFAD) is the by-product from oil refining coming from a distillation process used to eliminate free fatty acid from the oil.

The feedstock may be an intermediate product, a waste product or a by-product of oil or fat refining selected from the group consisting of: soap stock; acid oil; fatty acid distillates such as PFAD, soy fatty acid distillate, rapeseed fatty acid distillate, rice bran fatty acid distillate, poultry fat fatty acid distillate, beef tallow fatty acid distillate, etc.; gums from degumming; by-products from the production of omega-3 fatty acids derivates from fish oil; fat trap grease; yellow grease, and brown grease, free fatty acids like oleic acid; or fractions of oil obtained by physical separations; or any combinations thereof.

Free fatty acids (FFA): A free fatty acid is a carboxylic acid with a long carbon chain. Most naturally occurring fatty acids have an unbranched chain of an even number of carbon atoms, from 4 to 24. Free fatty acids are usually derived from fats (triglycerides (TAG), diglycerides (DAG), monoglyceride(MAG)), phospholipids or lyso-phospholipids. Triglycerides are formed by combining glycerol with three fatty acid molecules. The hydroxyl (HO—) group of glycerol and the carboxyl (—COOH) group of the fatty acid join to form an ester. The glycerol molecule has three hydroxyl (HO—) groups. Each fatty acid has a carboxyl group (—COOH). Diglycerides are formed by combining glycerol with two fatty acid molecules. Monoglycerides are formed by combining glycerol with one fatty acid molecule.

### DETAILED DESCRIPTION OF THE INVENTION

Biodiesel represents a promising alternative fuel for use in compression-ignition (diesel) engines. The biodiesel standards require or indirectly specify that biodiesel should be fatty acid methyl esters (FAME). However, we will use the term biodiesel broadly for fatty acid alkyl esters of short-chain alcohols obtained by the following reaction: Glycerides+FFA+alcohol→fatty acid alkyl ester (biodiesel)+glycerol+water. A short-chain alcohol is an alcohol having 1 to 5 carbon atoms (C₁-C₅). A preferred short-chain alcohol is ethanol or methanol.

The present invention relates to a process for producing fatty acid alkyl esters. The process comprises steps of: a) providing a substrate comprising triglycerides, diglycerides, monoglycerides, free fatty acids, or any combination thereof, and b) reacting the substrate with an enzyme composition comprising an sn-1,3 position lipase and an sn-2 position lipase to produce fatty acid alkyl esters.

In one aspect, the enzyme composition comprises a sn-1,3 position lipase, wherein the lipase has at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1 and sn-2 position lipase, wherein the lipase has at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 3, wherein said lipases being in liquid form.

In another aspect, the present invention further comprises reduction of free fatty acids in the produced fatty acid alkyl esters in step b) by saponification and/or esterification in presence of an enzyme or acid followed by separation and optionally recovering glycerol.

In one aspect of the invention, alcohol and optionally water are added during step b) of the process.

In one aspect of the invention, the alcohol is a C1-C5 alcohol, preferably methanol, ethanol, propanol, or mixtures thereof.

Excess of alcohol may drive the equilibrium reaction towards full conversion. For the purpose of the present invention the amount of alcohol is stated in equivalents (eq.) that is molar ratio of methanol to fatty acid (FA) present in the substrate (MetOH:FA) whether it is glycerol-bound FA or free FA.

In certain embodiments the present invention relates to a process of producing fatty acid ethyl esters, wherein the molar ratio of methanol to fatty acid in the substrate (MetOH:FA) is about 1.0-4.0 molar equivalents, such as 1.3-3.5 molar equivalents, such as 1.5-2.5 equivalents.

Proteins are generally destabilized in the presence of short-chain alcohols such as methanol and ethanol and inactivation of lipolytic enzymes occurs rapidly upon contact with insoluble alcohol, which exists as drops in the oil. Accordingly, it is often recommended that the amount of alcohol is kept below its solubility limits in oil. This may be obtained by a continuous and/or step-wise addition of alcohol.

In one aspect of the invention, the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.

The substrate comprising triglyceride is mixed with alcohol, preferably methanol or ethanol and heated to 30-60° C., preferably 40° C. on a reciprocal water shaking bath (200 rpm). Preferably water is added, and the solution is mixed and further heated to the desired temperature. The enzyme composition is added, and the solution is mixed vigorously and left on reciprocal water shaking bath at the desired temperature, preferably 40° C. and 200 rpm to react. The phases of the reaction mixture can be mixed by the use of high shear mixers, such as types from Silverson or IKA Labortechnik, as used in enzymatic degumming of vegetable oil (Clausen, K. (2001), European Journal of Lipid Science and Technology, vol. 103, 333-340).

In certain embodiments the present invention relates to a process for producing fatty acid ethyl esters, wherein methanol is added continuously and/or step-wise.

The alcohol can be added stepwise and/or continuously to the reaction over time. Water can be added separately or within an aqueous enzyme composition solution. The final concentration of water in the reaction mixture can be 0-50% (w/w), preferably 0-10%, more preferably 0.5-5%. The substrate comprises 0.00001-100% (w/w) triglyceride. Further, the substrate may comprise free fatty acids amounting to 0-100% (w/w). Also, mono- and diglycerides and phospholipids may be present (higher glyceride content of feedstock better the process efficiency).

Depending on the total amount of methanol to be used in the conversion reaction the number of steps in step-wise addition may vary. Thus, step-wise addition may constitute at least 1 step, at least 2 steps; at least 3 steps; at least 4 steps; at least 5 steps; at least 6 steps; at least 7 steps; at least 8 steps; at least 9 steps; or at least 10 steps.

Lipolytic enzymes are in general rather thermostable in oils, and the commercial process for enzymatic interesterification is generally performed at 70° C. Short-chain alcohols, however, have a negative impact on the stability and accordingly the activity of lipolytic enzymes and this destabilizing effect increases with increasing temperature. The destabilizing effect of alcohols on lipolytic enzymes seems to decrease with increasing alcohol molecular weight. The connection between solubility of the alcohol in oil and the destabilizing effect of the oil has been noted by several groups.

A few cases have described a positive effect of high alcohol dosage: In situations where the enzyme is very robust or if a larger alcohol without inactivating properties is used inactivation is not a problem. In that case the high alcohol concentration may be an advantage to drive the equilibrium reaction to full conversion.

Full conversion of a triglyceride-substrate results in formation of glycerol as a byproduct. Glycerol has been shown to inactivate especially immobilized lipolytic enzymes, presumably by physically blocking the access of substrate to the enzyme. It has been suggested that high alcohol concentrations may help avoiding that glycerol inactivate lipolytic enzymes by keeping the glycerol in solution. It has been shown that adsorbed glycerol on used silica particles may be removed by ethanol followed by drying (“Near-quantitative production of fatty acid alkyl esters by lipase-catalyzed alcoholysis of fats and oils with adsorption of glycerol by silica gel” Stevenson et al. (1994) Enzyme Microb. Technol., vol. 16, p. 478-484).

It has repeatedly been pointed out that the presence of water is important to maintain the activity of the lipolytic enzyme, and the majority of currently known methods prescribe addition of water to the reaction. It has surprisingly been found that the method of the present invention may be performed without additional water.

The process of the invention can be applied to any oil or fat consisting largely of triglycerides (triacylglycerol), e.g., vegetable oils and animal fat, typically containing more than 90 percent (e.g., more than 95 percent) by weight of triglycerides. The acyl groups in the triglyceride may be linear fatty acyl groups, typically with 4-24 carbon atoms, particularly 12-22 carbon atoms.

They may be saturated or unsaturated containing one or more double bonds. The triglyceride may particularly be a triglyceride of unsubstituted acyl groups, i.e., acyl groups of the general formula R—CO where R is a hydrocarbyl group. The process can be conducted at moderate temperatures, and advantageously it can even be applied to thermolabile triglycerides, e.g., triglycerides with polyunsaturated acyl groups. The process leads to a high yield of glycerol and fatty acid which can be separated and purified by conventional methods.

In one aspect of the invention, the process is perfomed in a batch or continuous mode.

The process may be carried out by contacting the substrate in a stirred tank with an enzyme composition in native (soluble) form.

The stirred tank may be used batchwise or continuously. The effluent from the stirred tank (whether batchwise or continuous) may be separated into an oily phase containing triglyceride and fatty acid, and an aqueous phase containing glycerol and lipase. Water and lipase from the aqueous phase may be recycled, optionally after separation of glycerol, for better utilization of the lipase.

Typical conditions for stirred-tank operation are 30-60 degrees centigrade, particularly 40-55 degrees centigrade, and a reaction time from 6 to 72 hours, particularly 12 to 48 hours.

In one aspect of the invention, at least 80%, at least 85%, at least 90% or such as at least 95% of the fatty acid acyl groups or free fatty acids in said substrate have been converted to fatty acid alkyl esters.

In one aspect of the invention, the total duration of step b) is from 5-72 hours, such as 10-70 hours, such as 12-68 hours, such as 18-60 hours, such as 24-48 hours in a batch process.

In one aspect of the invention, the total duration of step b) is from 5-80 hours, such as 10-75 hours, such as 12-72 hours, such as 18-60 hours, such as 24-48 hours in a continuous process.

In one aspect of the invention, the optionally the amount of water added in step b) is in the range 0.01%-10% of total substrate.

### Enzyme Composition

The invention uses two lipolytic enzymes or lipases (triacylglycerol lipase), i.e., enzymes that catalyze the hydrolysis of ester bonds in triglycerides (triacylglycerol). They are classified as EC 3.1.1.3 according to Enzyme Nomenclature. The two lipases are characterized by their positional specificity, i.e., the specificity for acyl groups in the 3 positions of a triglyceride. Thus, the microbial positionally specific (or 1,3-specific) lipase hydrolyzes acyl groups in the 1- and 3-positions with little or no activity in the 2-position, whereas the positionally non-specific lipase hydrolyzes acyl groups in all three positions at comparable rates. The positional specificity of a lipase may be determined as described in WO8802775, in WO 8901032 or in Example 8 of WO 9414940. The present invention uses a positionally non-specific lipase and a positional specific lipase. Each lipase may be used in native (soluble) form or in immobilized form.

In the context of this invention lipolytic enzymes are classified in E. C. 3.1.1 and include true lipases, esterases, phospholipases, and lyso-phospholipases. More specifically the lipolytic enzyme may be a lipase as classified by EC 3.1.1.3, EC 3.1.1.23 and/or EC 3.1.1.26, an esterase as classified by EC 3.1.1.1, EC 3.1.1.2, EC 3.1.1.6, EC 3.1.1.7, and/or EC 3.1.1.8, a phospholipase as classified by EC 3.1.1.4 and/or EC 3.1.1.32, a lyso-phospholipase as classified by EC 3.1.1.5 and a cutinase as classified in EC 3.1.1.74. The two or more lipolytic enzyme may also be a mixture of two or more lipases. The two or more lipolytic enzyme may include a lipase and a phospholipase. The two or more lipolytic enzyme includes a lipase of EC 3.1.1.3. The two or more lipolytic enzyme includes a lipase having activity on tri-, di-, and monoglycerides. The two or more lipolytic enzyme includes an sn-1,3 position lipase and an sn-2 position lipase.

In one aspect, the invention relates to an enzyme composition for producing fatty acid alkyl esters comprising an sn-1,3 position lipase and an sn-2 position lipase.

The lipolytic enzyme preferably is of microbial origin, in particular of bacterial, of fungal or of yeast origin. In a particular embodiment, the lipolytic enzyme used may be derived from a strain of Absidia, in particular Absidia blakesleena and Absidia corymbifera, a strain of Achromobacter, in particular Achromobacter iophagus, a strain of Aeromonas, a strain of Alternaria, in particular Alternaria brassiciola, a strain of Aspergillus, in particular Aspergillus niger and Aspergillus flavus, a strain of Achromobacter, in particular Achromobacter iophagus, a strain of Aureobasidium, in particular Aureobasidium pullulans, a strain of Bacillus, in particular Bacillus pumilus, Bacillus strearothermophilus and Bacillus subtilis, a strain of Beauveria, a strain of Brochothrix, in particular Brochothrix thermosohata, a strain of Candida, in particular Candida cylindracea (Candida rugosa), Candida paralipolytica, Candida tsukubaensis, Candida auriculariae, Candida humicola, Cadida foliarum, Candida cylindracea (Cadida rugosa) and Candida antarctica, a strain of Chromobacter, in particular Chromobacter viscosum, a strain of Coprinus, in particular Coprinus cinerius, a strain of Fusarium, in particular Fusarium oxysporum, Fusarium solani, Fusarium solani pisi, and Fusarium roseum culmorum, a strain of Geotricum, in particular Geotricum penicillatum, a strain of Hansenula, in particular Hansenula anomala, a strain of Humicola, in particular Humicola brevispora, Humicula lanuginosa, Humicola brevis var. thermoidea, and Humicola insolens, a strain of Hyphozyma, a strain of Lactobacillus, in particular Lactobacillus curvatus, a strain of Metarhizium, a strain of Mucor, a strain of Paecilomyces, a strain of Penicillium, in particular Penicillium cyclopium, Penicillium crustosum and Penicillium expansum, a strain of Pseudomonas in particular Pseudomonas aeruginosa, Pseudomonas alcaligenes, Pseudomonas cepacia (syn. Burkholderia cepacia), Pseudomonas fluorescens, Pseudomonas fragi, Pseudomonas maltophilia, Pseudomonas mendocina, Pseudomonas mephitica lipolytica, Pseudomonas alcaligenes, Pseudomonas plantari, Pseudomonas pseudoalcaligenes, Pseudomonas putida, Pseudomonas stutzeri, and Pseudomonas wisconsinensis, a strain of Rhizoctonia, in particular Rhizoctonia solani, a strain of Rhizomucor, in particular Rhizomucor miehei, a strain of Rhizopus, in particular Rhizopus japonicus, Rhizopus microsporus and Rhizopus nodosus, a strain of Rhodosporidium, in particular Rhodosporidium toruloides, a strain of Rhodotorula, in particular Rhodotorula glutinis, a strain of Sporobolomyces, in particular Sporobolomyces shibatanus, a strain of Thermomyces, in particular Thermomyces lanuginosus (formerly Humicola lanuginosa), a strain of Thiarosporella, in particular Thiarosporella phaseolina, a strain of Trichoderma, in particular Trichoderma harzianum, and Trichoderma reesei, and/or a strain of Verticillium.

In one aspect of the invention, the sn-1,3 position lipase and the sn-2 position lipase is of microbial origin, in particular of fungal or bacterial origin.

In one aspect of the invention, the sn-1,3 position lipase and the sn-2 position lipase is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei, Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucorjavanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase.

### Sn-1,3 Position Lipase:

In a preferred embodiment, the sn-1,3 position lipase of the invention comprise a lipase derived from a strain of Thermomyces, in particular a strain of Thermomyces lanuginosus (TLL) (synonym Humicola lanuginosa) or a variant thereof. In a specific embodiment, the sn-1,3 position lipase is the one shown in SEQ ID NO: 1 or a variant thereof.

In an embodiment, the sn-1,3 position lipase is:
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1;
ii) a variant of a parent lipase having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 1;
iii) a fragment of the lipase in (i) or (ii) having lipase activity, wherein the variant comprises substitutions at positions corresponding to T231R+N233R and at least one or more (e.g., several) of D96E, D111A, D254S, G163K, P256T, G91T and G38A of SEQ ID NO: 1.

In a specific embodiment, the sn-1,3 position lipase, used in the invention, is a variant of a parent lipase, wherein the variant has lipase activity, has at least 60%, in particular at least 65%, at least 70%, at least 75% at least 80%, at least 85%, at least 90%, at least 95%, at least 96%, at least 97%, at least 98%, at least 99% but less than 100% sequence identity with SEQ ID NO: 1, and comprises substitutions selected from the group of:
D96E+T231R+N233R;
N33Q+D96E+T231R+N233R;
N33Q+D111A+T231R+N233R;
N33Q+T231R+N233R+P256T;
N33Q+G38A+G91T+G163K+T231R+N233R+D254S;
N33Q+G38A+G91T+D96E+D111A+G163K+T231R+N233R+D254S+P256T;
D27R+N33Q+G38A+D96E+D111A+G163K+T231R+N233R+D254S+P256T;
D27R+N33Q+G38A+G91T+D96E+D111A+G163K+T231R+N233R+P256T;
D27R+N33Q+G38A+G91T+D96E+D111A+G163K+T231R+N233R+D254S;
D27R+G38A+G91T+D96E+D111A+G163K+T231R+N233R+D254S+P256T;
D96E+T231R+N233R+D254S;
T231R+N233R+D254S+P256T;
G163K+T231R+N233R+D254S;
D27R+N33Q+G38A+G91T+D96E+G163K+T231R+N233R+D254S+P256T;
D27R+G91T+D96E+D111A+G163K+T231R+N233R+D254S+P256T;
D96E+G163K+T231R+N233R+D254S;
D27R+G163K+T231R+N233R+D254S;
D27R+G38A+G91T+D96E+D111A+G163K+T231R+N233R+D254S;
D27R+G38A+G91T+D96E+G163K+T231R+N233R+D254S+P256T;
D27R+G38A+D96E+D111A+G163K+T231R+N233R+D254S+P256T;
D27R+D96E+G163K+T231R+N233R+D254S;
D27R+D96E+D111A+G163K+T231R+N233R+D254S+P256T;
D27R+G38A+D96E+G163K+T231R+N233R+D254S+P256T
D111A+G163K+T231R+N233R+D254S+P256T;
D111A+T231R+N233R;
D111A+T231R+N233R+D254S+P256T;
D27R+D96E+D111A+G163K+T231R+N233R;
D27R+D96E+D111A+T231R+N233R;
D27R+N33Q+G38A+D96E+D111A+T231R+N233R+D254S+P256T;
D27R+G38A+D96E+D111A+G163K+E210Q+T231R+N233R+D254S+P256T;
D27R+T231R+N233R+D254S+P256T;
D96E+D111A+G163K+T231R+N233R;
D96E+D111A+G163K+T231R+N233R+D254S+P256T;
D96E+D111A+G163K+T231R+N233R+P256T;
D96E+D111A+T231R+N233R;
D96E+D111A+T231R+N233R+D254S;
D96E+D111A+T231R+N233R+D254S+P256T
D96E+D111A+T231R+N233R+P256T;
D96E+G163K+T231R+N233R+D254S+P256T;
D96E+T231R+N233R+D254S+P256T;
D96E+T231R+N233R+P256T;
G38A+D96E+D111A+T231R+N233R;
G91T+D96E+D111A+G163K+T231R+N233R+D254S+P256T;
G91T+D96E+D111A+T231R+N233R;
G91T+D96E+T231R+N233R;
G91T+T231R+N233R+D254S+P256T;
N33Q+D96E+D111A+G163K+T231R+N233R+D254S+P256T;
T231R+N233R+D254S+P256T;
T231R+N233R+P256T.

In another embodiment, the sn-1,3 position lipase is a variant of a parent lipase, wherein said variant
(a) comprises a modification in at least one position corresponding to positions E1, V2, N33, F51, E56, L69, K98, V176, H198, E210, Y220, L227, and K237 of SEQ ID NO: 1; and optionally further comprises a modification in at least one position corresponding to positions D27, G38, D96, D111, G163, T231, N233, D254, and P256 of SEQ ID NO: 1;
(b) has a sequence identity of at least 60%, at least 65%, at least 70%, at least 75% at least 80%, at least 85%, at least 90%, at least 95%, at least 96%, at least 97%, at least 98%, at least 99% but less than 100% to SEQ ID NO: 1;
(c) has lipase activity.

In an embodiment, the sn-1,3 position lipase is a variant of a parent lipase, wherein the parent lipase is selected from the group consisting of:
a) a polypeptide having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1;
b) a fragment of the polypeptide of SEQ ID NO: 1.

In an embodiment, the sn-1,3 position lipase is a variant having lipase activity and having at least 60%, at least 65%, at least 70%, at least 75%, at least 80%, at least 85%, at least 90%, at least 95% identity, at least 96%, at least 97%, at least 98%, or at least 99%, but less than 100% sequence identity to SEQ ID NO: 1.

In an embodiment, the sn-1,3 position lipase variant comprises a modification in at least one of the following positions corresponding to: E1, V2, D27, N33, G38, F51, E56, L69, D96, K98, D111, G163, V176, H198, E210, Y220, L227, T231, N233, K237, D254, and P256, wherein numbering is according to SEQ ID NO: 1. More preferably, the lipase variant comprises at least one of the following modifications corresponding to: E1C, V2Y, D27R, N33K, N33Q, G38A, F51V, E56K, L69R, D96E, D96L, K981, K98Q, D111A, G163K, V176L, H198S, E210K, Y220F, L227G, T231R, N233R, N233C, K237C, D254S, and P256T, wherein numbering is according to SEQ ID NO: 1.

In an embodiment, the said lipase variant further comprises one of the substitutions selected from the group of: S54T, S83T, G91A, A150G, I255A, and E239C.

In a preferred embodiment, the sn-1,3 position lipase variant comprises substitutions corresponding to E1C+N233C in SEQ ID NO: 1 and optionally one or more additional substitutions.

In a specific embodiment, the variant has lipase activity, has at least 60%, at least 65%, at least 70%, at least 75% at least 80%, at least 85%, at least 90%, at least 95%, at least 96%, at least 97%, at least 98%, at least 99% but less than 100% sequence identity with SEQ ID NO: 1 and comprises or consists of substitutions corresponding to one of the following set of substitutions using SEQ ID NO: 1 for numbering:

E1C + H198L + N233C
E1C + H198G + N233C
E1C + L69V + N233C
E1C + L69T + N233C
E1C + L69S + N233C
E1C + L69H + N233C
E1C + L69F + N233C
E1C + L69C + N233C
E1C + H198Y + N233C
E1C + H198T + N233C
E1C + H198G + N233C
E1C + L227F + N233C
E1C + L227R + N233C
E1C + E210T + N233C
E1C + E210N + N233C
E1C + V176M + N233C
E1C + K98T + N233C
E1C + K98E + N233C
E1C + E56S + N233C
E1C + E56Q + N233C
E1C + E56R + N233C
E1C + F51M + N233C
E1C + D27R + F51Y + N233C
E1C + V2I + N233C
E1C + V2N + N233C
E1C + V2K + N233C
E1C + V2A + N233C
E1C + D96L + N233C
E1C + L69R + N233C
E1C + V2Y + N233C
E1C + N233C + P256T
E1C + N233C + D254S
E1C + T231R + N233C
E1C + H198S + N233C
E1C + D111A + N233C
E1C + D96E + N233C
E1C + G38A + N233C
E1C + N33Q + N233C
E1C + N33K + N233C
E1C + E210A + N233C
E1C + E210Q + N233C
E1C + E210R + N233C
E1C + H198D + N233C
E1C + K98R + N233C
E1C + K98V + N233C
E1C + F51L + N233C
E1C + F51I + N233C
E1C + K237C
E1C + L227G + N233C
E1C + E210K + N233C
E1C + V176L + N233C
E1C + K98Q + N233C
E1C + E56K + N233C
E1C + L147S + N233C + D254S
E1C + Y220F + N233C
E1C + K98I + N233C
E1C + N233C
E1C + D27R + F51I + E56R + K98E + T231R + N233C
E1C + D27R + F51I + E56R + K98E + T231R + N233C + D254S
E1C + D27R + G38A + F51L + K98I + D111A + G163S + H198S + Y220F + T231R + N233C + P256T
E1C + D27R + G38A + F51L + D96E + K98I + D111A + G163K + H198S + Y220F + T231R + N233C +
D254S + P256T
E1C + D27R + G38R + F51L + D96E + K98I + D111A + G163K + H198S + Y220F + T231R + N233C +
D254S + P256T
E1C + D27R + F51L + D96I + K98I + D111A + G163K + H198S + Y220F + T231R + N233C + P256T
E1C + D27R + F51L + D96E + K98I + D111A + G163S + H198S + Y220F + T231R + N233C + P256T
E1C + D27R + F51L + D96E + K98I + D111A + G163K + H198S + Y220F + T231R + N233C + P256T
E1C + D27R + G38A + F51I + D96E + K98I + D111A + G163K + H198S + Y220F + T231R + N233C +
D254S + P256T
E1C + D27R + G38A + F51V + D96E + K98I + D111A + G163K + H198S + Y220F + T231R + N233C +
P256T
E1C + D27R + F51V + D96E + K98I + D111A + G163K + H198S + Y220F + T231R + N233C + P256T
E1C + D27R + F51V + D96E + K98I + D111A + G163S + H198S + Y220F + T231R + N233C + D254S +
P256T
E1C + D27R + F51V + D96I + K98I + D111A + G163K + H198S + Y220F + T231R + N233C + D254S +
P256T
E1C + D27R + F51V + K98I + D111A + G163K + H198S + Y220F + T231R + N233C + D254S + P256T
E1C + D27R + F51V + D96E + K98I + D111A + G163K + H198S + Y220F + T231R + N233C + D254S +
P256T
E1C + D27R + G38A + F51V + D96E + K98I + D111A + G163K + H198S + Y220F + T231R + N233C +
D254S + P256T
E1C + F51V + D96E + K98I + D111A + G163S + H198S + Y220F + T231R + N233C + P256T
E1C + F51L + D96E + K98I + D111A + G163K + H198S + Y220F + T231R + N233C + D254S + P256T
E1C + G38A + F51V + D96E + K98I + D111A + G163K + H198S + Y220F + T231R + N233C + P256T
E1C + F51V + D96E + K98I + D111A + G163K + H198S + Y220F + T231R + N233C + P256T
E1C + F51V + D96I + K98I + D111A + G163K + H198S + Y220F + T231R + N233C + D254S + P256T
E1C + F51V + K98I + D111A + G163K + H19S + Y220F + T231R + N233C + D254S + P256T
E1C + F51I + D96E + K98I + D111A + G163K + H198S + Y220F + T231R + N233C + D254S + P256T
E1C + D27R + F51L + D96E + K98I + D111A + G163K + H198S + Y220F + T231R + N233C + D254S +
P256T
E1C + D27R + N33K + G38A + F51V + D96E + K98I + D111A + G163K + H198S + Y220F + T231R + N233C
E1C + G38R + F51V + D96E + K98I + D111A + G163K + H198S + Y220F + T231R + N233C + D254S +
P256T
E1C + F51V + D96E + K98I + D111A + G163K + H198S + Y220F + T231R + N233C + D254S + P256T
E1C + G38A + F51V + D96E + K98I + D111A + G163K + H198S + Y220F + T231R + N233C + D254S +
P256T
E1C + D27R + G38R + F51V + D96E + K98I + D111A + G163K + H198S + Y220F + T231R + N233C +
D254S + P256T

Sn-2 lipase: The sn-2 lipase may be microbial, e.g., fungal or bacterial, particularly one derived from the following genera and species as described in the indicated publications: Candida, C. rugosa (also called C. cylindracea), C. antarctica lipase A or B (WO 8802775), Pseudomonas, P. cepacia (WO 8901032), Streptomyces (WO 9414940). It may also be a variant obtained by substitution, deletion or insertion of one or more amino acids in of one of the indicated lipases, e.g. as described in WO 9401541.

In a preferred embodiment the sn-2 lipase has at least 60%, such as at least 70%, such as at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, such as 100% identity to the mature part of the polypeptide of SEQ ID NO: 3 or SEQ ID NO: 4 herein, preferably derived from a strain of the genus Moesziomyces, such as a strain of Moesziomyces antarcticus (Candida antarcticus).

In an embodiment the sn-2 lipase comprises or consists of the amino acid sequence of SEQ ID NO: 3 or SEQ ID NO: 4, or an allelic variant thereof; or is a fragment thereof having sn-2 position lipase activity. In another embodiment, the sn-2 position lipase comprises or consists of the mature polypeptide of SEQ ID NO: 3 or SEQ ID NO: 4, or a variant of the mature polypeptide of SEQ ID NO: 3 or SEQ ID NO: 4 comprising a substitution, deletion, and/or insertion at one or more positions.

In a more preferred embodiment, the sn-2 lipase used according to the invention is derived from a strain of Bacillus pumilus, a strain of Bacillus stearothermophilus a strain of Candida cylindracea, a strain of Candida antarctica, in particular Candida antarctica Lipase A (obtained as described in WO 88/02775)

Commercial lipase preparations suitable for use in the process of the invention include LIPOZYME® TL 100L, CALLERA™ TRANS and Eversa® Transform, Eversa® Transform 2.0, Novocor ADL (all available from Novozymes A/S), or mixtures thereof.

### Lipase Activity:

In the context of the present invention, the lipolytic activity may be determined as lipase units (LU), using tributyrate as substrate. The method is based on the hydrolysis of tributyrin by the enzyme, and the alkali consumption to keep pH constant during hydrolysis is registered as a function of time

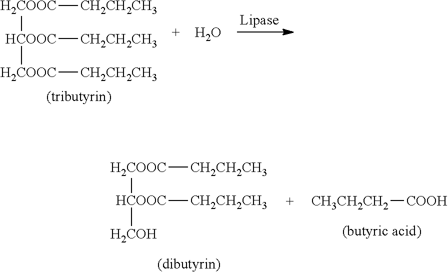

One lipase unit (LU) may be defined as the amount of enzyme which, under standard conditions (i.e. at 30° C.; pH 7.0; with 0.1% (w/v) Gum Arabic as emulsifier and 0.16 M tributyrine as substrate) liberates 1 micromol titrable butyric acid per minute.

Alternatively, lipolytic activity may be determined as Long Chain Lipase Units (LCLU) using substrate pNP-Palmitate (C:16) when incubated at pH 8.0, 30° C., the lipase hydrolyzes the ester bond and releases pNP, which is yellow and can be detected at 405 nm.

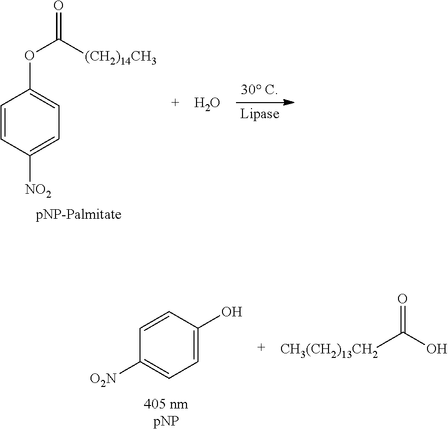

### Lipolytic Enzyme Dosage

Enzyme dosage is for the purpose of the present invention expressed as the percentage weight/weight (% w/w) of lipolytic enzyme added to the reaction mixture with respect to the substrate. For example, 10% enzyme in 100 g oil would translate to 10 g enzyme added. Although an increased amount of lipolytic enzyme in general reduces the conversion time, it is desirable from an economic point of view to operate at reduced levels of enzyme dosage.

In certain embodiments the present invention relates to a process for producing fatty acid ethyl esters, wherein the enzyme composition is dosed about 0.01-1.0 g enzyme protein (EP)/kg of substrate.

Enzyme sources and formulation: The two or more lipolytic enzyme used in the process of the invention may be derived or obtainable from any of the sources mentioned herein. The term “derived” means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence. Within the meaning of a native enzyme are included natural variants. Furthermore, the term “derived” includes enzymes produced synthetically by e.g., peptide synthesis. The term “derived” also encompasses enzymes which have been modified e.g., by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g., peptide synthesis. With respect to recombinantly produced enzyme the terms “obtainable” and “derived” refers to the identity of the enzyme and not the identity of the host organism in which it is produced recombinantly.

Accordingly, the two or more lipolytic enzyme may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The two or more lipolytic enzyme may be applied in any suitable formulation, e.g., as lyophilized powder or in liquid/aqueous solution.

Further, the invention relates to a batch process and/or a continuous, staged process to produce fatty acid alkyl esters using a first and a second lipolytic enzyme as described above, wherein the alcohol is added continuously and/or stepwise, and wherein the enzymes are recycled or used only once. If the enzymes are in an aqueous phase, this phase can be separated from the fatty phase by a decanter, a settler or by centrifugation. In the continuously process the two phases, oil and aqueous, respectively, can be processed counter-currently.

### Cloning a DNA Sequence Encoding a Lipolytic Enzyme

The DNA sequence encoding a parent lipolytic enzyme may be isolated from any cell or microorganism producing the lipolytic enzyme in question, using various methods well known in the art. First, a genomic DNA and/or cDNA library should be constructed using chromosomal DNA or messenger RNA from the organism that produces the lipolytic enzyme to be studied. Then, if the amino acid sequence of the lipolytic enzyme is known, labelled oligonucleotide probes may be synthesized and used to identify lipolytic enzyme-encoding clones from a genomic library prepared from the organism in question. Alternatively, a labelled oligonucleotide probe containing sequences homologous to another known lipolytic enzyme gene could be used as a probe to identify lipolytic enzyme-encoding clones, using hybridization and washing conditions of lower stringency.

Yet another method for identifying lipolytic enzyme-encoding clones would involve inserting fragments of genomic DNA into an expression vector, such as a plasmid, transforming cutinase-negative bacteria with the resulting genomic DNA library, and then plating the transformed bacteria onto agar containing a substrate for lipolytic enzyme (i.e. triglyceride), thereby allowing clones expressing the lipolytic enzyme to be identified.

Alternatively, the DNA sequence encoding the enzyme may be prepared synthetically by established standard methods, e.g. the phosphoroamidite method described by S. L. Beaucage and M. H. Caruthers, (1981), Tetrahedron Letters 22, p. 1859-1869, or the method described by Matthes et al., (1984), EMBO J. 3, p. 801-805. In the phosphoroamidite method, oligonucleotides are synthesized, e.g. in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or R. K. Saiki et al., (1988), Science 239, 1988, pp. 487-491.

### Expression Vector

The recombinant expression vector carrying the DNA sequence encoding a lipolytic enzyme of the invention may be any vector which may conveniently be subjected to recombinant DNA procedures, and the choice of vector will often depend on the host cell into which it is to be introduced. The vector may be one which, when introduced into a host cell, is integrated into the host cell genome and replicated together with the chromosome(s) into which it has been integrated. Examples of suitable expression vectors include pMT838.

The expression vector of the invention may also comprise a suitable transcription terminator and, in eukaryotes, polyadenylation sequences operably connected to the DNA sequence encoding the lipolytic enzyme of the invention. Termination and polyadenylation sequences may suitably be derived from the same sources as the promoter.

The vector may further comprise a DNA sequence enabling the vector to replicate in the host cell in question. Examples of such sequences are the origins of replication of plasmids pUC19, pACYC177, pUB110, pE194, pAMB1 and pIJ702.

The vector may also comprise a selectable marker, e.g. a gene the product of which complements a defect in the host cell, such as the dal genes from B. subtilis or B. licheniformis, or one which confers antibiotic resistance such as ampicillin, kanamycin, chloramphenicol or tetracyclin resistance. Furthermore, the vector may comprise Aspergillus selection markers such as amdS, argB, niaD and sC, a marker giving rise to hygromycin resistance, or the selection may be accomplished by co-transformation, e.g. as described in WO 91/17243.

The procedures used to ligate the DNA construct of the invention encoding a cutinase variant, the promoter, terminator and other elements, respectively, and to insert them into suitable vectors containing the information necessary for replication, are well known to persons skilled in the art (cf., for instance, Sambrook et al., Molecular Cloning: A Laboratory Manual, 2nd Ed., Cold Spring Harbor, 1989).

### Promoter

In the vector, the DNA sequence should be operably connected to a suitable promoter sequence. The promoter may be any DNA sequence which shows transcriptional activity in the host cell of choice and may be derived from genes encoding proteins either homologous or heterologous to the host cell.

Examples of suitable promoters for directing the transcription of the DNA sequence encoding a lipolytic enzyme of the invention, especially in a bacterial host, are the promoter of the lac operon of E. coli, the Streptomyces coelicolor agarase gene dagA promoters, the promoters of the Bacillus licheniformis alfa-amylase gene (amyL), the promoters of the Bacillus stearothermophilus maltogenic amylase gene (amyM), the promoters of the Bacillus amyloliquefaciens alfa-amylase (amyQ), the promoters of the Bacillus subtilis xylA and xylB genes etc. For transcription in a fungal host, examples of useful promoters are those derived from the gene encoding A. oryzae TAKA amylase, the TPI (triose phosphate isomerase) promoter from S. cerevisiae (Alber et al. (1982), J. Mol. Appl. Genet 1, p. 419-434, Rhizomucor miehei aspartic proteinase, A. niger neutral alfa-amylase, A. niger acid stable alfa-amylase, A. nigerglucoamylase, Rhizomucor miehei lipase, A. oryzae alkaline protease, A. oryzae triose phosphate isomerase, or A. nidulans acetamidase.

### Host Cells

The cell of the invention, either comprising a DNA construct or an expression vector of the invention as defined above, is advantageously used as a host cell in the recombinant production of a lipolytic enzyme of the invention. The cell may be transformed with the DNA construct of the invention encoding the lipolytic enzyme, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g. by homologous or heterologous recombination. Alternatively, the cell may be trans-formed with an expression vector as described above in connection with the different types of host cells.

The cell of the invention may be a cell of a higher organism such as a mammal or an insect, particularly a microbial cell, e.g. a bacterial or a fungal (including yeast) cell.

Examples of suitable bacteria are Gram positive bacteria such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, or Streptomyces lividans or Streptomyces murinus, or gram negative bacteria such as E. coli. The transformation of the bacteria may, for instance, be effected by protoplast transformation or by using competent cells in a manner known per se.

The yeast organism may favorably be selected from a species of Saccharomyces or Schizosaccharomyces, e.g. Saccharomyces cerevisiae.

The host cell may also be a filamentous fungus e.g. a strain belonging to a species of Aspergillus, particularly Aspergillus oryzae or Aspergillus niger, or a strain of Fusarium, such as a strain of Fusarium oxysporum, Fusarium graminearum (in the perfect state named Gibberella zeae, previously Sphaeria zeae, synonym with Gibberella roseum and Gibberella roseum f. sp. cerealis), or Fusarium sulphureum (in the prefect state named Gibberella puricaris, synonym with Fusarium trichothecioides, Fusarium bactridioides, Fusarium sambucinum, Fusarium roseum, and Fusarium roseum var. graminearum), Fusarium cerealis (synonym with Fusarium crokkwellense), or Fusarium venenatum.

In a particular embodiment of the invention the host cell is a protease deficient or protease minus strain. This may for instance be the protease deficient strain Aspergillus oryzae JaL 125 having the alkaline protease gene named “alp” deleted. This strain is described in WO 97/35956 (Novo Nordisk).

Filamentous fungi cells may be transformed by a process involving protoplast formation and transformation of the protoplasts followed by regeneration of the cell wall in a manner known per se. The use of Aspergillus as a host microorganism is described in EP 238 023 (Novo Nordisk A/S), the contents of which are hereby incorporated by reference.

### Production of Lipolytic Enzyme by Cultivation of Transformant

The invention relates, inter alia, to a method of producing a lipolytic enzyme of the invention, which method comprises cultivating a host cell under conditions conducive to the production of the lipolytic enzyme and recovering the lipolytic enzyme from the cells and/or culture medium.

The medium used to cultivate the cells may be any conventional medium suitable for growing the host cell in question and obtaining expression of the lipolytic enzyme of the invention. Suitable media are available from commercial suppliers or may be prepared according to published recipes (e.g., as described in catalogues of the American Type Culture Collection).

The lipolytic enzyme secreted from the host cells may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography, or the like.

### Enzymatic Biodiesel Process Design

The process setup is very important as it has to take into account technical issues, such as homogeneity of reaction/product mixture, solubility of alcohol, stability of enzyme, recovery of enzyme, etc. There are several different process designs to be considered: batch and continuous stirred tank reactors. These will briefly be outlined in the following paragraphs.

The batch process is a typical process used in the laboratory due to the simple setup. This process can be operated with addition of all components from the start, i.e., in bulk, or with step-wise and/or continuous addition of alcohol which is recommended. The batch process is useful in collecting data about the process, such as productivity of the enzyme. Negative elements of this process setup in large scale are the large tank volume required, the long reaction time, and the fact that this process is not continuous. Another very important fact to consider is the gradual decline in enzyme activity as the number of re-uses increase. When the enzyme activity decreases, the reaction time must be increased accordingly to keep a constant degree of conversion or additional enzyme can be added to make up for the activity loss.

A continuous stirred tank reactor is a container with a continuous supply of feed and withdrawal of product. The design requires multiple tanks in series to assure the same degree of conversion for the same reaction time, meaning the total tank volume will likely increase relative to a similar batch system because of an increased total residence time requirement in the continuous system. An advantage of such a continuous system is that the capacity of the plant is typically increased because time required for emptying/filling is eliminated. Another advantage of a continuous design is the possibility of introducing separation steps between the tanks such as to continuously eliminate the glycerol formed. Additionally, a continuous system will reach a steady state, where most process parameters remain fairly constant assuming constant input parameters. This allows for tuning of a continuous system in a way that allows for optimum operation of the enzyme by providing steady state operating conditions that outperform those of an evolving batch system which will naturally change operating conditions, especially component concentrations, through the duration of reaction.

In certain embodiments the present invention relates to a process for producing fatty acid ethyl esters, wherein said process is selected from the group of process designs containing: batch, continuous stirred-tank reactor, and others.

### Feed Stocks for Enzymatic Production of Biodiesel

Fatty acid ethyl esters may be prepared from several types of vegetable oils. In the global vegetable oil production palm oil is leading the gains and has the highest yield compared to that of other vegetable oils, and it would therefore be economically intuitive to consider palm oil as a favorable feed stock for biodiesel production. One may, however, argue in favor of using inedible oils such as Jatropha oil, as edible oils are not in surplus supply. Examples of plants which may serve as feed stock for vegetable oils for use as substrate in the production of fatty acid ethyl esters are such as babassu, borage, canola, coconut, corn, cotton, hemp, jatropha, karanj, mustard, palm, peanut, rapeseed, rice, soybean, and sunflower.

Microalgae is also considered as feed stock in the production of biodiesel due to the higher photosynthetic efficiency of microalgae in comparison with plants and hence a potentially higher productivity per unit area.

Alternatively, fatty acid ethyl esters may be prepared from non-vegetable feed stocks like animal fat such as lard, tallow, butterfat and poultry or marine oils.

It has been estimated that 60-90% of the biodiesel cost arises from the cost of the feed stock oil, and thus use of cheaper waste oil would have a great impact in reducing the cost of biodiesel. In addition, it is considered an important step in reducing and recycling waste oil. Fresh vegetable oil and its waste differ in their content of water and free fatty acid. Unlike the conventional chemical routes for synthesis of diesel fuels, biocatalytic routes permit one to carry out the transesterification of a wide variety of oil feed stocks in the presence of acidic impurities, such as free fatty acids. Accordingly, fatty acid distillates (from deodorizer/fatty acid stripping), acid oils (from soap stock splitting in chemical oil refining), waste oils and used oils may serve as feed stock in the production of biodiesel.

Thus, the feed stock can be of crude quality or further processed (refined, bleached and deodorized). Suitable oils and fats may be pure triglyceride or a mixture of triglyceride, diglyceride, monoglyceride, and free fatty acids, commonly seen in waste vegetable oil and animal fats. The feed stock may also be obtained from vegetable oil deodorizer distillates. The type of fatty acids in the feed stock comprises those naturally occurring as glycerides in vegetable and animal fats and oils. These include oleic acid, linoleic acid, linolenic acid, palmetic acid, steric acid, and lauric acid to name a few. Minor constituents in crude vegetable oils are typically phospholipids, free fatty acids and partial glycerides i.e., mono- and diglycerides.

In certain embodiments the present invention relates to a process for producing fatty acid alkyl esters, wherein the substrate is selected from the group containing: babassu oil; borage oil; canola oil; coconut oil; corn oil; cotton oil; hemp oil; jatropha oil; karanj oil; mustard oil; palm oil; peanut oil; rapeseed oil; rice oil; soybean oil; and sunflower oil; oil from microalgae; animal fat; tallow; lard; butterfat; poultry; marine oils; tuna oil; fatty acid distillates; acid oils; waste oil; used oil; partial glycerides and any combinations thereof.

### Re-Use of Lipolytic Enzyme Composition in the Production of Fatty Acid Ethyl Esters

In certain embodiments the present invention relates to re-use of enzyme composition in the production of fatty acid ethyl esters obtained by reacting methanol with a substrate comprising triglyceride, diglyceride, monoglyceride; free fatty acids or any combination thereof, wherein the molar ratio of methanol to fatty acid in the substrate (MetOH:FA) is about 1.0-4.0 equivalents; the enzyme dosage is below 1% w/w (enzyme protein to oil) with respect to the substrate; and which enzyme after use in a conversion reaction is separated from the resulting reaction mixture and re-used directly without modifications in the next conversion reaction. By modification is meant any treatment or activity such as activation, washing, drying etc. apart from the separation of the lipolytic enzyme from the reaction mixture.

In certain embodiments the present invention relates to re-use of enzyme composition in the production of fatty acid ethyl esters, wherein the sn-1,3 position lipase and sn-2 position lipase is selected from the group consisting of is selected from the group containing: Thermomyces lanuginosa lipase; Candida Antarctica B lipase; Candida deformans lipase; Candida lipolytica lipase; Candida parapsilosis lipase; Candida rugosa lipase; Cryptococcus spp. S-2 lipase; Rhizomucor miehei lipase; Rhizomucor delemar lipase; Burkholderia (Pseudomonas) cepacia lipase; Pseudomonas camembertii lipase; Pseudomonas fluorescens lipase; Geotrichium candidum lipase; Hyphozyma sp. lipase; Klebsiella oxytoca lipase; and variants thereof.

In certain embodiments the present invention relates to re-use of enzyme composition in the production of fatty acid ethyl esters, wherein the enzyme composition is dosed within the range of 0.01-1.0 g enzyme protein (EP)/kg of substrate.

In certain embodiments the present invention relates to re-use of the enzyme composition in the production of fatty acid ethyl esters, wherein methanol is added continuous or step-wise.

In certain embodiments the present invention relates to re-use of the enzyme composition in the production of fatty acid ethyl esters, wherein said process is selected from the group of process designs containing: batch, continuous stirred-tank reactor, and others.

In certain embodiments the present invention relates to re-use of at least of the enzyme composition in the production of fatty acid ethyl esters, wherein the substrate is selected from the group containing: babassu oil; borage oil; canola oil; coconut oil; corn oil; cotton oil; hemp oil; jatropha oil; karanj oil; mustard oil; palm oil; peanut oil; rapeseed oil; rice oil; soybean oil; and sunflower oil; oil from microalgae; animal fat; tallow; lard; butterfat; poultry; marine oils; tuna oil; fatty acid distillates; acid oils; waste oil; used oil; partial glycerides and any combinations thereof.

In certain embodiments of the invention, the reuse of the enzyme composition is facilitated by recirculation of all or part of the heavy phase resulting from the reaction. Especially in case of liquid enzyme composition formulations, the enzyme composition will almost entirely be found in the heavy phase and can be reused through separation of light and heavy phase such as through gravitational settling principles such as centrifugation or decantation.

The invention is described in the following numbered paragraphs:
1. A process for producing fatty acid alkyl esters comprising steps:
a) providing a substrate comprising triglycerides, diglycerides, monoglycerides, free fatty acids, or any combination thereof, and
b) reacting the substrate with an enzyme composition comprising an sn-1,3 position lipase and an sn-2 position lipase to produce fatty acid alkyl esters.
2. The process of paragraph 1, further comprising reduction of free fatty acids in the produced fatty acid alkyl esters in step b) by saponification and/or esterification in presence of an enzyme or acid followed by separation and optionally recovering glycerol.
3. The process according to any of the preceding paragraphs, wherein alcohol and optionally water are added during step b).
4. The process according to any of the preceding paragraphs, wherein said alcohol is a C1-C5 alcohol, preferably methanol, ethanol, propanol, or mixtures thereof.
5. The process according to any of the preceding paragraphs, wherein the molar ratio of methanol to fatty acid in the substrate (MetOH:FA) is about 1.0-4.0 molar equivalents, such as 1.3-3.5 molar equivalents, such as 1.5-2.5 equivalents.
6. The process according to any of the preceding paragraphs, wherein the process is proceeding in a batch or continuous mode.
7. The process according to any of the preceding paragraphs, wherein the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.
8. The process according to any of the preceding paragraphs, wherein the total duration of step b) is from 5-72 hours, such as 10-70 hours, such as 12-68 hours, such as 18-60 hours, such as 24-48 hours in a batch process.
9. The process according to any of the preceding paragraphs, wherein the total duration of step b) is from 5-80 hours, such as 10-75 hours, such as 12-72 hours, such as 18-60 hours, such as 24-48 hours in a continuous process.
10. The process according to any of the preceding paragraphs, wherein optionally the amount of water added in step b) is in the range 0.01%-10% of total substrate.
11. The process according to any of the preceding paragraphs, wherein at least 80%, at least 85%, at least 90% or such as at least 95% of the fatty acid acyl groups or free fatty acids in said substrate have been converted to fatty acid alkyl esters.
12. An enzyme composition for producing fatty acid alkyl esters comprising an sn-1,3 position lipase and an sn-2 position lipase.
13. The composition of paragraph 12, wherein the sn-1,3 position lipase and the sn-2 position lipase is of microbial origin, in particular of fungal or bacterial origin.
14. The composition of any one of paragraphs 12-13, wherein the sn-1,3 position lipase and the sn-2 position lipase is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei, Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucorjavanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase.
15. The composition of any one of paragraphs 12-14, wherein the sn-1,3 position lipase is
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1;
ii) a variant of a parent lipase having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 1;
iii) a fragment of the lipase in (i) or (ii) having lipase activity.
16. The composition of any one of paragraphs 12-15, wherein the sn-1,3 position lipase is a variant comprises substitutions at positions corresponding to T231R+N233R and optionally at least one or more (e.g., several) of D96E, D111A, D254S, G163K, P256T, G91T and G38A of SEQ ID NO: 1.
17. The composition of any one of paragraphs 12-16, wherein the sn-1,3 position lipase is a variant of a parent lipase, wherein the variant has lipase activity, has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1, and comprises substitutions at positions corresponding to T231R+N233R and at least one or more (e.g., several) of D96E, D111A, D254S, G163K, P256T, G91T and G38A of SEQ ID NO: 1 selected from the group of:
   a. D96E+T231R+N233R;
   b. N33Q+D96E+T231R+N233R;
   c. N33Q+D111A+T231R+N233R;
   d. N33Q+T231R+N233R+P256T;
   e. N33Q+G38A+G91T+G163K+T231R+N233R+D254S;
   f. N33Q+G38A+G91T+D96E+D111A+G163K+T231R+N233R+D254S+P256T;
   g. D27R+N33Q+G38A+D96E+D111A+G163K+T231R+N233R+D254S+P256T;
   h. D27R+N33Q+G38A+G91T+D96E+D111A+G163K+T231R+N233R+P256T;
   i. D27R+N33Q+G38A+G91T+D96E+D111A+G163K+T231R+N233R+D254S;
   j. D27R+G38A+G91T+D96E+D111A+G163K+T231R+N233R+D254S+P256T;
   k. D96E+T231R+N233R+D254S;
   l. T231R+N233R+D254S+P256T;
   m. G163K+T231R+N233R+D254S;
   n. D27R+N33Q+G38A+G91T+D96E+G163K+T231R+N233R+D254S+P256T;
   o. D27R+G91T+D96E+D111A+G163K+T231R+N233R+D254S+P256T;
   p. D96E+G163K+T231R+N233R+D254S;
   q. D27R+G163K+T231R+N233R+D254S;
   r. D27R+G38A+G91T+D96E+D111A+G163K+T231R+N233R+D254S;
   s. D27R+G38A+G91T+D96E+G163K+T231R+N233R+D254S+P256T;
   t. D27R+G38A+D96E+D111A+G163K+T231R+N233R+D254S+P256T;
   u. D27R+D96E+G163K+T231R+N233R+D254S;
   v. D27R+D96E+D111A+G163K+T231R+N233R+D254S+P256T;
   w. D27R+G38A+D96E+G163K+T231R+N233R+D254S+P256T
   x. D111A+G163K+T231R+N233R+D254S+P256T;
   y. D111A+T231R+N233R;
   z. D111A+T231R+N233R+D254S+P256T;
   aa. D27R+D96E+D111A+G163K+T231R+N233R;
   bb. D27R+D96E+D111A+T231R+N233R;
   cc. D27R+N33Q+G38A+D96E+D111A+T231R+N233R+D254S+P256T;
   dd. D27R+G38A+D96E+D111A+G163K+E210Q+T231R+N233R+D254S+P256T;
   ee. D27R+T231R+N233R+D254S+P256T;
   ff. D96E+D111A+G163K+T231R+N233R;
   gg. D96E+D111A+G163K+T231R+N233R+D254S+P256T;
   hh. D96E+D111A+G163K+T231R+N233R+P256T;
   ii. D96E+D111A+T231R+N233R;
   jj. D96E+D111A+T231R+N233R+D254S;
   kk. D96E+D111A+T231R+N233R+D254S+P256T
   ll. D96E+D111A+T231R+N233R+P256T;
   mm. D96E+G163K+T231R+N233R+D254S+P256T;
   nn. D96E+T231R+N233R+D254S+P256T;
   oo. D96E+T231R+N233R+P256T;
   pp. G38A+D96E+D111A+T231R+N233R;
   qq. G91T+D96E+D111A+G163K+T231R+N233R+D254S+P256T;
   rr. G91T+D96E+D111A+T231R+N233R;
   ss. G91T+D96E+T231R+N233R;
   tt. G91T+T231R+N233R+D254S+P256T;
   uu. N33Q+D96E+D111A+G163K+T231R+N233R+D254S+P256T;
   vv. T231R+N233R+D254S+P256T;
   ww. T231R+N233R+P256T.
18. The composition of any one of paragraphs 12-17, wherein the sn-1,3 position lipase is a variant of a parent lipase, wherein said variant
(a) comprises a modification in at least one position corresponding to positions E1, V2, N33, F51, E56, L69, K98, V176, H198, E210, Y220, L227, and K237 of SEQ ID NO: 1; and optionally further comprises a modification in at least one position corresponding to positions D27, G38, D96, D111, G163, T231, N233, D254, and P256 of SEQ ID NO: 1;
(b) has a sequence identity of at least 60% but less than 100% to SEQ ID NO: 1;
(c) has lipase activity.
19. The composition of any one of paragraphs 12-18, wherein said sn-1,3 position lipase variant comprises a modification in at least one of the following positions: E1, V2, D27, N33, G38, F51, E56, L69, D96, K98, D111, G163, V176, H198, E210, Y220, L227, T231, N233, K237, D254, and P256, wherein numbering is according to SEQ ID NO: 1.
20. The composition of any one of paragraphs 18 or 19, wherein said sn-1,3 position lipase variant comprises at least one of the following modifications: E1C, V2Y, D27R, N33K, N33Q, G38A, F51V, E56K, L69R, D96E, D96L, K981, K98Q, D111A, G163K, V176L, H198S, E210K, Y220F, L227G, T231R, N233R, N233C, K237C, D254S, and P256T, wherein numbering is according to SEQ ID NO: 1.
21. The composition of any one of paragraphs 18-20, wherein said sn-1,3 position lipase variant further comprises one of the substitutions selected from the group of: S54T, S83T, G91A, A150G, I255A, and E239C, wherein numbering is according to SEQ ID NO: 1.
22. The composition of any one of paragraphs 18-21, wherein the sn-1,3 position lipase variant comprises substitutions E1C+N233C and one or more additional substitutions, wherein numbering is according to SEQ ID NO: 1.
23. The composition of any one of paragraphs 18-22, wherein the sn-1,3 position lipase has lipase activity, has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1 and comprises or consists of substitutions corresponding to one of the following set of substitutions using SEQ ID NO: 1 for numbering

E1C + H198L + N233C
E1C + H198G + N233C
E1C + L69V + N233C
E1C + L69T + N233C
E1C + L69S + N233C
E1C + L69H + N233C
E1C + L69F + N233C
E1C + L69C + N233C
E1C + H198Y + N233C
E1C + H198T + N233C
E1C + H198G + N233C
E1C + L227F + N233C
E1C + L227R + N233C
E1C + E210T + N233C
E1C + E210N + N233C
E1C + V176M + N233C
E1C + K98T + N233C
E1C + K98E + N233C
E1C + E56S + N233C
E1C + E56Q + N233C
E1C + E56R + N233C
E1C + F51M + N233C
E1C + D27R + F51Y + N233C
E1C + V2I + N233C
E1C + V2N + N233C
E1C + V2K + N233C
E1C + V2A + N233C
E1C + D96L + N233C
E1C + L69R + N233C
E1C + V2Y + N233C
E1C + N233C + P256T
E1C + N233C + D254S
E1C + T231R + N233C
E1C + H198S + N233C
E1C + D111A + N233C
E1C + D96E + N233C
E1C + G38A + N233C
E1C + N33Q + N233C
E1C + N33K + N233C
E1C + E210A + N233C
E1C + E210Q + N233C
E1C + E210R + N233C
E1C + H198D + N233C
E1C + K98R + N233C
E1C + K98V + N233C
E1C + F51L + N233C
E1C + F51I + N233C
E1C + K237C
E1C + L227G + N233C
E1C + E210K + N233C
E1C + V176L + N233C
E1C + K98Q + N233C
E1C + E56K + N233C
E1C + L147S + N233C + D254S
E1C + Y220F + N233C
E1C + K98I + N233C
E1C + N233C
E1C + D27R + F51I + E56R + K98E + T231R + N233C
E1C + D27R + F51I + E56R + K98E + T231R + N233C + D254S
E1C + D27R + G38A + F51L + K98I + D111A + G163S + H198S + Y220F + T231R + N233C + P256T
E1C + D27R + G38A + F51L + D96E + K98I + D111A + G163K + H198S + Y220F + T231R + N233C +
D254S + P256T
E1C + D27R + G38R + F51L + D96E + K98I + D111A + G163K + H198S + Y220F + T231R + N233C +
D254S + P256T
E1C + D27R + F51L + D96I + K98I + D111A + G163K + H198S + Y220F + T231R + N233C + P256T
E1C + D27R + F51L + D96E + K98I + D111A + G163S + H198S + Y220F + T231R + N233C + P256T
E1C + D27R + F51L + D96E + K98I + D111A + G163K + H198S + Y220F + T231R + N233C + P256T
E1C + D27R + G38A + F51I + D96E + K98I + D111A + G163K + H198S + Y220F + T231R + N233C +
D254S + P256T
E1C + D27R + G38A + F51V + D96E + K98I + D111A + G163K + H198S + Y220F + T231R + N233C +
P256T
E1C + D27R + F51V + D96E + K98I + D111A + G163K + H198S + Y220F + T231R + N233C + P256T
E1C + D27R + F51V + D96E + K98I + D111A + G163S + H198S + Y220F + T231R + N233C + D254S +
P256T
E1C + D27R + F51V + D96I + K98I + D111A + G163K + H198S + Y220F + T231R + N233C + D254S +
P256T
E1C + D27R + F51V + K98I + D111A + G163K + H198S + Y220F + T231R + N233C + D254S + P256T
E1C + D27R + F51V + D96E + K98I + D111A + G163K + H198S + Y220F + T231R + N233C + D254S +
P256T
E1C + D27R + G38A + F51V + D96E + K98I + D111A + G163K + H198S + Y220F + T231R + N233C +
D254S + P256T
E1C + F51V + D96E + K98I + D111A + G163S + H198S + Y220F + T231R + N233C + P256T
E1C + F51L + D96E + K98I + D111A + G163K + H198S + Y220F + T231R + N233C + D254S + P256T
E1C + G38A + F51V + D96E + K98I + D111A + G163K + H198S + Y220F + T231R + N233C + P256T
E1C + F51V + D96E + K98I + D111A + G163K + H198S + Y220F + T231R + N233C + P256T
E1C + F51V + D96I + K98I + D111A + G163K + H198S + Y220F + T231R + N233C + D254S + P256T
E1C + F51V + K98I + D111A + G163K + H19S + Y220F + T231R + N233C + D254S + P256T
E1C + F51I + D96E + K98I + D111A + G163K + H198S + Y220F + T231R + N233C + D254S + P256T
E1C + D27R + F51L + D96E + K98I + D111A + G163K + H198S + Y220F + T231R + N233C +
D254S + P256T
E1C + D27R + N33K + G38A + F51V + D96E + K98I + D111A + G163K + H198S + Y220F +
T231R + N233C
E1C + G38R + F51V + D96E + K98I + D111A + G163K + H198S + Y220F + T231R + N233C +
D254S + P256T
E1C + F51V + D96E + K98I + D111A + G163K + H198S + Y220F + T231R + N233C + D254S + P256T
E1C + G38A + F51V + D96E + K98I + D111A + G163K + H198S + Y220F + T231R + N233C +
D254S + P256T
E1C + D27R + G38R + F51V + D96E + K98I + D111A + G163K + H198S + Y220F + T231R +
N233C + D254S + P256T.

24. The composition of paragraphs 12-23, wherein the sn-1,3 position lipase is
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 2;
ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 2;
iii) a fragment of the lipase in (i) or (ii) having lipase activity.
25. The composition of paragraph 12, wherein the sn-2 position lipase is
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 3;
ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 3;
iii) a fragment of the lipase in (i) or (ii) having lipase activity.
26. The composition according to any of the preceding paragraphs 12-25, wherein the total amount of said enzyme composition is within the range of 0.01-1.0 g enzyme protein (EP)/kg of substrate.
27. Use of an enzyme composition of any one of paragraphs 12-26 for producing fatty acid alkyl esters.

### EXAMPLES

### Example 1: Testing the Combination of Eversa Transform (ET) 2.0 (SEQ ID NO: 1) in Combination with Novocor ADL (CaIA Formulation) (SEQ ID NO: 3). Both Enzymes from Novozymes, Denmark

30 g crude palm oil (CPO) or Crude Palm Kernel Oil (CPKO), NaOH (10 ppm for CPO and 50 ppm for CPKO), 2.5% water, MeOH 1.7 eqv, 0.3 eqv from start and 1.4 during 20 h.

### Enzyme Composition:

1: 0,1% ET2.0 HS,
2: 0,08% ET2.0 HS+CalA eqv to 0,02% ET2.0,
3: 0,06% ET2.0 HS+CalA eqv to 0,04% ET2.0 HS.
NB the low enzyme dosage 0.1% ET2.0 HS.

Samples are made in doublets. 40° C., shaking incubator 250 rpm. Sampling at 1, 3, 20 and 24 h respectively. Analyze for AG, relative glyceride composition, analyzed on QTA from Eurofins by calibration B-100 (method according to AOCS Method CK 2-09) (on 24 h samples), 24 h samples FFA % measured by titration. Samples are dried in speed vacuum.
ET HS 3.9% active enzyme-protein (39 mg/mL)
Novocor (CaIA) AL 1.65% active enzyme-protein.
0.243% Novocor (CaIA) ADL=0.1% ET2.0 HS

QTA B100

| | MG, % | DG, % | TG, % | FAME, % |
|---|---|---|---|---|
| Sample 24 h | Average | Average | Average | Average |
| 1: CPO - 0.1% ET2.0 HS | 6.0 | 3.1 | 1.9 | 74.0 |
| 2: CPO - 0.08% ET2.0 HS + | 4.8 | 2.9 | 1.8 | 77.3 |
| CalA eqv to 0.02% ET2.0 | | | | |
| 3: CPO - 0.06% ET2.0 HS + | 4.7 | 2.7 | 1.6 | 78.5 |
| CalA eqv to 0.04% ET2.1 | | | | |
| 4: CPKO - 0.1% ET2.0 HS | 7.88 | >5.5 | Outlier | *87.53 |
| 5: CPKO - 0.08% ET2.0 HS + | 6.226 | 4.987 | Outlier | *92.72 |
| CalA eqv to 0.02% ET2.0 | | | | |
| 6: CPKO - 0.06% ET2.0 HS + | 9.666 | Outlier | Outlier | *80.51 |
| CalA eqv to 0.04% ET2.1 | | | | |

FFA, %

| | FFA, % |
|---|---|
| Sample 24 h | Average |
| 1: CPO - 0.1% ET2.0 HS | 2.3 |
| 2: CPO - 0.08% ET2.0 HS + CalA eqv to 0.02% ET2.0 | 2.1 |
| 3: CPO - 0.06% ET2.0 HS + CalA eqv to 0.04% ET2.1 | 2.2 |
| 4: CPKO - 0.1% ET2.0 HS | 3.5 |
| 5: CPKO - 0.08% ET2.0 HS + CalA eqv to 0.02% ET2.0 | 3.3 |
| 6: CPKO - 0.06% ET2.0 HS + CalA eqv to 0.04% ET2.1 | 4.1 |

The results indicated a better conversion with increasing CalA substitution for GPO, and for CPKO only the 20% substitution showed better conversion whereas the 40% substitution was similar to pure ET.

### Example 2: Various Feedstocks with Emphasis on Total Glycerides Towards End of Reaction

30 g fatty acid feedstock of various qualities. CPKO and CPO being triglyceride oils of relatively high quality relative to POME and UCO being low quality feedstocks with typically high initial FFA levels.

High initial FFA translates to more water in the endpoint reaction mixture and therefore higher FFA at equilibrium.

2% water added with the enzyme mixture. The total enzyme protein in the mixture is similar in all cases and correspond to the amount of enzyme dosed using 0.2% ET2.0.
Measured concentrations using NIR (QTA, Eurofins, B100 method) at t=30 h
For CPKO and CPO a low MeOH dosage (1.6 eqv) and slow 20 h dosing rate was optimum and is reported below.
For POME and UCO a high MeOH dosage (2.2 eqv) and fast dosing rate of 10 hours was optimum and is reported below.

| CPKO | FAME | FFA | Total glycerdes |
|---|---|---|---|
| 100% ET | 100 | 2.6 | 2.8 |
| 80/20 ET/CalA | 100 | 2.5 | 1.5 |

NB: The QTA-method is known to have significant deviation for FAME on CPKO.

| CPO | FAME | FFA | Total glycerdes |
|---|---|---|---|
| 100% ET | 93.3 | 1.8 | 2.2 |
| 80/20 ET/CalA | 95.9 | 1.7 | 0.5 |

| POME | FAME | FFA | Total glycerdes |
|---|---|---|---|
| 100% ET | 90.4 | 3.8 | 0.4 |
| 80/20 ET/CalA | 90.7 | 3.9 | 0 |

| UCO | FAME | FFA | Total glycerdes |
|---|---|---|---|
| 100% ET | 88.6 | 3.2 | 0.9 |
| 80/20 ET/CalA | 88.8 | 3.2 | 0.5 |

Total glyceride levels are generally lowered using the ET/CALA mixture.

### Example 3: Comparison of SEQ ID NO: 4 Versus SEQ ID NO: 3

Standard biodiesel reaction with 2% water, 50 ppm sodium hydroxide. 1.7 equivalent methanol dosed over 20 h. 30 g CPO in 100 mL square bluecap flasks incubated at 35° C., 250 rpm shaking incubator. 0.2% of enzyme of SEQ ID NO: 1 as standardized enzyme protein mass dosage is 2.34 mg enzyme protein in total in all trials. 80/20 SEQ ID NO: 1/SEQ ID NO: 3 on protein mass basis or SEQ ID NO: 1/SEQ ID NO: 4 on protein mass basis. Samples are made in duplicate.

| Enzyme | # | Time | MG wt % | DG wt % | TG wt % |
|---|---|---|---|---|---|
| 100% SEQ ID NO: 1 | 1 | 20 h | 2.39 | 3.74 | 3.11 |
| 1.7 eqv MeOH/20 h | | 22 h | 2.35 | 1.29 | 1.00 |
| | | 24 h | 1.63 | 1.03 | 0.75 |
| 80% SEQ ID NO: 1/ | 1 | 20 h | 1.81 | 2.32 | 1.37 |
| 20% SEQ ID NO: 3 | | 22 h | 1.79 | 1.06 | 0.74 |
| 1.7 eqv MeOH/20 h | | 24 h | 1.30 | 0.86 | 0.65 |
| | 2 | 20 h | 2.35 | 1.34 | 1.11 |
| | | 22 h | 1.82 | 1.15 | 0.83 |
| | | 24 h | 1.38 | 0.83 | 0.59 |
| 80% SEQ ID NO: 1/ | 1 | 20 h | 3.12 | 1.82 | 1.25 |
| 20% SEQ ID NO: 4 | | 22 h | 2.46 | 1.48 | 1.01 |
| 1.7 eqv MeOH/20 h | | 24 h | 1.95 | 1.18 | 0.77 |
| | 2 | 20 h | 3.86 | 1.77 | 1.14 |
| | | 22 h | 2.69 | 1.69 | 1.07 |
| | | 24 h | 2.15 | 1.28 | 0.88 |

Considering the 20 h samples, which are furthest from equilibrium, it is evident that SEQ ID NO: 3 increases rate of reaction relative to SEQ ID NO: 1 alone, and that SEQ ID NO: 3 brings more improvement than SEQ ID NO: 4. Then, considering the 24 h samples, SEQ ID NO: 3 brings improvement relative to SEQ ID NO: 4 and SEQ ID NO: 1 Alone. SEQ ID NO: 1 alone outperforms SEQ ID NO: 4 with 24 h reaction time, meaning near equilibrium, where SEQ ID NO: 4 excellent esterification capabilities might be having a negative effect on the results. Therefore, SEQ ID NO: 3 is preferred.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-10. (canceled)

11: A process for producing fatty acid alkyl esters, said process comprising:
a) providing a substrate comprising triglycerides, diglycerides, monoglycerides, free fatty acids or any combination thereof, and
b) reacting the substrate with an enzyme composition comprising an sn-1,3 position lipase and an sn-2 position lipase to produce fatty acid alkyl esters;
wherein the sn-1,3 position lipase is a lipase having at least 80% sequence identity to SEQ ID NO: 1 or SEQ ID NO: 2;
wherein the sn-2 position lipase is a lipase having at least 80% sequence identity to SEQ ID NO: 3; and
wherein said lipases are in liquid form.

12: The process of claim 11, further comprising reduction of free fatty acids in the produced fatty acid alkyl esters in step b) by saponification and/or esterification in presence of an enzyme or acid followed by separation and optionally recovering glycerol.

13: The process of claim 11, wherein alcohol is added during step b).

14: The process of claim 13, wherein said alcohol is a C1-C5 alcohol.

15: The process of claim 13, wherein said alcohol is methanol, ethanol, propanol, or mixtures thereof.

16: The process of claim 11, wherein water is added during step b).

17: The process according to claim 11, wherein the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and animal fat.

18: The process according to claim 11, wherein at least 80% of the fatty acid acyl groups or free fatty acids in said substrate has been converted to fatty acid alkyl esters.

19: The process according to claim 11, wherein at least 95% of the fatty acid acyl groups or free fatty acids in said substrate has been converted to fatty acid alkyl esters.

20: The process according to claim 11, wherein the sn-1,3 position lipase is a lipase having at least 90% sequence identity to SEQ ID NO: 1 or SEQ ID NO: 2.

21: The process according to claim 11, wherein the sn-1,3 position lipase is a lipase having at least 95% sequence identity to SEQ ID NO: 1 or SEQ ID NO: 2.

22: The process according to claim 11, wherein the sn-1,3 position lipase is a lipase comprising an amino acid sequence of SEQ ID NO: 1 or SEQ ID NO: 2.

23: The process according to claim 11, wherein the sn-2 position lipase is a lipase having at least 90% sequence identity to SEQ ID NO: 3.

24: The process according to claim 11, wherein the sn-2 position lipase is a lipase having at least 95% sequence identity to SEQ ID NO: 3.

25: The process according to claim 11, wherein the sn-2 position lipase is a lipase comprising an amino acid sequence of SEQ ID NO: 3.

26: An enzyme composition comprising an sn-1,3 position lipase and an sn-2 position lipase,
wherein the sn-1,3 position lipase is a lipase having at least 80% sequence identity to SEQ ID NO: 1 or SEQ ID NO: 2;
wherein the sn-2 position lipase is a lipase having at least 80% sequence identity to SEQ ID NO: 3; and
wherein said lipases are in liquid form.
